(19) United States
(12) Patent Application Publication
(10) Pub. No.: US 2012/0187313 A1
(43) Pub. Date: Jul. 26, 2012

(54) Systems, Devices, and/or Methods for Managing Disinfection

(76) Inventors: Mike Clark, Columbia, VA (US); Michele Wells, Columbia, VA (US)

(21) Appl. No.: 13/355,346

(22) Filed: Jan. 20, 2012

Related U.S. Application Data
(60) Provisional application No. 61/434,553, filed on Jan. 20, 2011.

(51) Int. Cl. G21K 5/00; B23P 17/04

(52) U.S. Cl. 250/492.1; 29/428

(57) ABSTRACT

Certain exemplary embodiments can provide a system, which can comprise a mat comprising a plurality of ultraviolet lamps. The ultraviolet lamps can be adapted to disinfect a surface of the mat. In certain exemplary embodiments, the mat can be adapted to transfer heat from the ultraviolet lamps to a surrounding environment.

### CROSS-REFERENCES TO RELATED APPLICATIONS

This application claims priority to, and incorporates by reference herein in its entirety, pending U.S. Provisional Patent Application Ser. No. 61/434,553 (Attorney Docket No. 1017-001), filed 20 Jan. 2011.

### BRIEF DESCRIPTION OF THE DRAWINGS

A wide variety of potential embodiments will be more readily understood through the following detailed description of certain exemplary embodiments, with reference to the accompanying exemplary drawings in which:

FIG. 1 is a perspective view of an exemplary embodiment of a system; and

FIG. 2 is a flowchart of an exemplary embodiment of a method 2000.

### DETAILED DESCRIPTION

Certain exemplary embodiments provide a mat, which can comprise a plurality of ultraviolet light sources. The ultraviolet light sources can be adapted to function to disinfect one or more surfaces of the mat.

Ultraviolet (UV) light is electromagnetic radiation with a wavelength shorter than that of visible light, but longer than X-rays, in the range of approximately 10 nm to approximately 400 nm, and energies from approximately 3 eV to approximately 124 eV. Ultraviolet (UV) light energy comprises electromagnetic waves with frequencies higher than those that humans identify as the color violet. Ultraviolet is substantially invisible to the human eye. UV light is found in sunlight and can be emitted by electric arcs and/or specialized lights such as black lights. Some ultraviolet radiation can be classified as non-ionizing radiation, and can cause chemical reactions, and/or many substances to glow or fluoresce under it. Higher energies of the ultraviolet spectrum from approximately 150 nm can be ionizing.

Fluorescent lamps, without a phosphorescent coating to convert UV to visible light, can emit ultraviolet light with two peaks at approximately 254 nm and approximately 185 nm due to peak emissions of mercury within the bulb. Germicidal lamps can use quartz (glass) doped with an additive to substantially block radiation having a wavelength of approximately 185 nm. Low-pressure mercury lamps can be used for disinfection and can have an optimum operating temperature of approximately 30 degrees Celsius. Using a mercury amalgam can allow an operating temperature to rise to approximately 100 degrees Celsius.

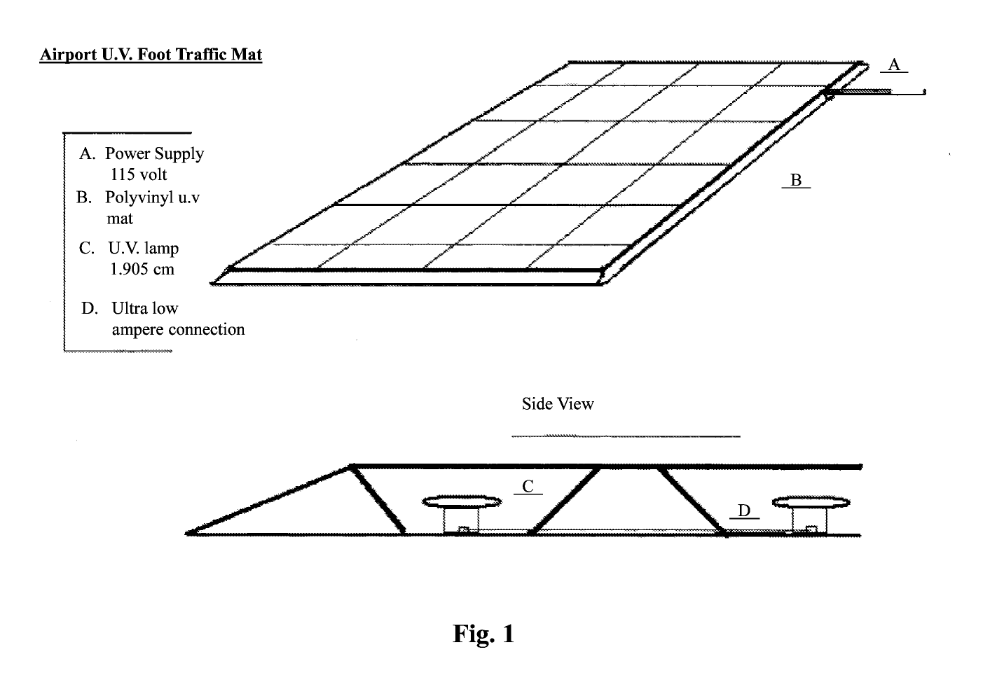

FIG. 1 is a perspective view of an exemplary embodiment of a system, which can comprise a mat having a cover that is translucent or substantially transparent to ultraviolet radiation. For example, the mat can be a material such as polyurethane or polyvinyl with ultraviolet radiation sources incorporated into the mat. The mat can be adapted to reduce bacteria, viral, and/or fungal growth on walking areas. In certain exemplary embodiments, the mat can be used at security check points, such as at airports, government buildings, courthouses, and/or jail facilities where persons are asked and/or required to remove their footwear. Walking on mat through such check point areas can reduce a probability of acquiring bacterial, fungal and viral infections compared to other alternatives.

In certain exemplary embodiments, the ultraviolet radiation sources can be ultraviolet lamps that comprise a bulb that is less than approximately 2 centimeters in diameter. Any of a wide variety of ultraviolet lamp bulb sizes can be used in exemplary embodiments. Electrical current draws to exemplary embodiments can be dependent upon the characteristics and size of the ultraviolet radiation sources that are utilized. Each of the ultraviolet radiation sources can be electrically coupled to an electrical energy source via an electrical conductor. Each of the ultraviolet radiation sources can comprise a base portion adapted to condition electrical energy from the electrical energy source for use by the ultraviolet radiation sources. In certain exemplary embodiments the electrical energy source can have an electrical voltage of approximately 115 alternating current volts.

Certain exemplary embodiments can provide structural members and/or sections adapted to restrain an upper layer of the mat from deflecting sufficiently to contact a surface of the ultraviolet radiation sources. Each of the structural members and/or sections can be adapted to maintain a distance of separation between a first layer and an opposing second layer of the mat. Each of the structural members and/or sections can be adapted to separate one or more pairs of the ultraviolet radiation sources. Each of the ultraviolet radiation sources can be adapted to be releasably or unreleasably coupled directly to the first layer of the mat. In certain exemplary embodiments, the structural members and/or sections can be sized and/or designed to allow the mat to have a soft and/or spongy feel to persons walking on the mat. In certain exemplary embodiments, the mat can comprise a non-skid surface such as comprising ridges and/or beads intrinsic to the mat.

In certain exemplary embodiments, an upper layer of the mat, a lower layer of the mat, and/or structural members and/or sections can be adapted to transfer heat generated by the ultraviolet radiation sources to a surrounding environment. Such heat transfer can be via conduction, convection, and/or radiation.

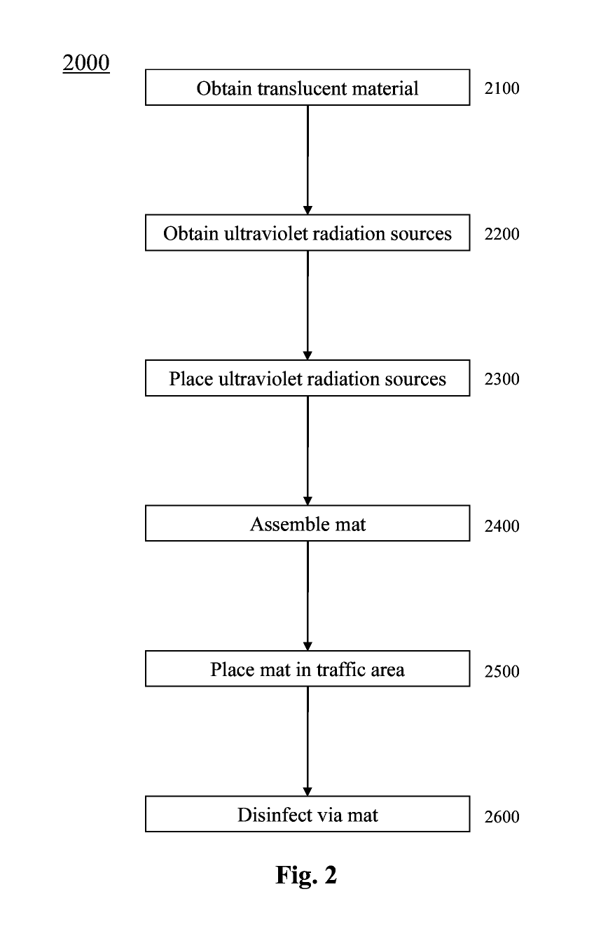

FIG. 2 is a flowchart of an exemplary embodiment of a method 2000. At activity 2100, a material can be obtained that is translucent and/or transparent to ultraviolet radiation.

At activity 2200, a plurality of ultraviolet radiation sources can be obtained. At activity 2300, the ultraviolet radiation sources can be placed in the mat.

At activity 2400, the mat can be assembled. At activity 2500, the mat can be placed in traffic area, such as at check points at airports, government buildings, courthouses, jails, prisons, public arenas, and/or any other area where people might be required to remove clothing such as shoes for security purposes.

At activity 2600, the mat can be used for disinfection purposes.

### Definitions

When the following terms are used substantively herein, the accompanying definitions apply. These terms and definitions are presented without prejudice, and, consistent with the application, the right to redefine these terms during the prosecution of this application or any application claiming priority hereto is reserved. For the purpose of interpreting a claim of any patent that claims priority hereto, each definition (or redefined term if an original definition was amended during the prosecution of that patent), functions as a clear and unambiguous disavowal of the subject matter outside of that definition.

a—at least one.

activity—an action, act, step, and/or process or portion thereof.

adapted to—made suitable or fit for a specific use or situation.

adapter—a device used to effect operative compatibility between different parts of one or more pieces of an apparatus or system.

and/or—either in conjunction with or in alternative to.

apparatus—an appliance or device for a particular purpose

associate—to join, connect together, and/or relate.

can—is capable of, in at least some embodiments.

circuit—an electrically conductive pathway and/or a communications connection established across two or more switching devices comprised by a network and between corresponding end systems connected to, but not comprised by the network.

comprising—including but not limited to.

configure—to make suitable or fit for a specific use or situation.

connect—to join or fasten together.

coupleable—capable of being joined, connected, and/or linked together.

coupling—linking in some fashion.

define—to establish the outline, form, or structure of.

determine—to obtain, calculate, decide, deduce, and/or ascertain.

device—a machine, manufacture, and/or collection thereof.

generate—to create, produce, give rise to, and/or bring into existence.

install—to connect or set in position and prepare for use.

may—is allowed and/or permitted to, in at least some embodiments.

method—a process, procedure, and/or collection of related activities for accomplishing something.

plurality—the state of being plural and/or more than one.

predetermined—established in advance.

provide—to furnish, supply, give, and/or make available.

set—a related plurality.

store—to place, hold, and/or retain data, typically in a memory.

substantially—to a great extent or degree.

support—to bear the weight of, especially from below.

system—a collection of mechanisms, devices, machines, articles of manufacture, processes, data, and/or instructions, the collection designed to perform one or more specific functions.

via—by way of and/or utilizing.

### Note

Still other substantially and specifically practical and useful embodiments will become readily apparent to those skilled in this art from reading the above-recited and/or herein-included detailed description and/or drawings of certain exemplary embodiments. It should be understood that numerous variations, modifications, and additional embodiments are possible, and accordingly, all such variations, modifications, and embodiments are to be regarded as being within the scope of this application.

Thus, regardless of the content of any portion (e.g., title, field, background, summary, description, abstract, drawing figure, etc.) of this application, unless clearly specified to the contrary, such as via explicit definition, assertion, or argument, with respect to any claim, whether of this application and/or any claim of any application claiming priority hereto, and whether originally presented or otherwise:
there is no requirement for the inclusion of any particular described or illustrated characteristic, function, activity, or element, any particular sequence of activities, or any particular interrelationship of elements;
no characteristic, function, activity, or element is “essential”;
any elements can be integrated, segregated, and/or duplicated;
any activity can be repeated, any activity can be performed by multiple entities, and/or any activity can be performed in multiple jurisdictions; and
any activity or element can be specifically excluded, the sequence of activities can vary, and/or the interrelationship of elements can vary.

Moreover, when any number or range is described herein, unless clearly stated otherwise, that number or range is approximate. When any range is described herein, unless clearly stated otherwise, that range includes all values therein and all subranges therein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

When any claim element is followed by a drawing element number, that drawing element number is exemplary and non-limiting on claim scope. No claim of this application is intended to invoke paragraph six of 35 USC 112 unless the precise phrase “means for” is followed by a gerund.

Any information in any material (e.g., a United States patent, United States patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such material is specifically not incorporated by reference herein.

Accordingly, every portion (e.g., title, field, background, summary, description, abstract, drawing figure, etc.) of this application, other than the claims themselves, is to be regarded as illustrative in nature, and not as restrictive, and the scope of subject matter protected by any patent that issues based on this application is defined only by the claims of that patent.

What is claimed is:

1. A system comprising:
a mat comprising a plurality of ultraviolet lamps, said ultraviolet lamps adapted to disinfect a surface of said mat.

2. The system of claim 1, wherein the mat is adapted to transfer heat from said ultraviolet lamps to a surrounding environment.

3. The system of claim 1, wherein said mat comprises a non-slip surface.

4. A method comprising a plurality of activities, comprising:
fabricating a mat, said mat comprising a plurality of ultraviolet lamps.

5. The method of claim 4, further comprising placing said mat in an airport.

6. The method of claim 4, wherein said mat comprises a plurality of support gussets, said support gussets adapted to restrain motion of a layer of said mat relative to surfaces of said ultraviolet lamps.
